(19) United States
(12) Patent Application Publication
(10) Pub. No.: US 2025/0186658 A1
(43) Pub. Date: Jun. 12, 2025

(54) PROCESS FOR PREPARING A DRIED, SWELLABLE, SPONGY BIOLOGICAL MATERIAL FOR IMPLANTS

(71) Applicant: BIOTRONIK AG, Buelach (CH)

(72) Inventors: Alexander Rzany, Nuernberg (DE); Nina Foh, Forchheim (DE); Bernhard Hensel, Erlangen (DE)

(21) Appl. No.: 18/847,414

(22) PCT Filed: Apr. 6, 2023
(86) PCT No.: PCT/EP2023/059180

§ 371 Date: Sep. 16, 2024

(30) Foreign Application Priority Data
Apr. 29, 2022 (EP) 22170917.3

(51) Int. Cl. A61L 27/56; A61L 24/00; A61L 27/36

(52) U.S. Cl. CPC A61L 27/56; A61L 24/0005; A61L 24/0036; A61L 27/3604; A61L 27/3687; A61L 27/3691; A61L 2400/04

(57) ABSTRACT

A method for preparing a biological material for a medical application includes exposing the biological material to a first liquid, for example including, by at least 10%, a component other than water, and applying a first compression step onto the biological material, wherein the first compression step includes repeatedly applying a compression force in a plurality of compression intervals. The method further includes drying the biological material, and can included applying second and/or third liquids after respective first and second compression steps.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/059180, which was filed Apr. 6, 2023, which application claimed priority from EP Application Serial No. 22170917.3, which was filed Apr. 29, 2022.

### FIELD OF THE INVENTION

A field of the invention concerns the making of swellable biological materials. Such materials have utility, for example, as part of an implant that is used to seal and stop blood flow.

### BACKGROUND

A variety of medical interventions require a stopping of undesired bleedings. Such medical interventions, e.g., include dental surgeries in which a sponge-like material may be placed in a bleeding region of a Cavitas oris of a patient. The sponge-like material may preferably be adapted such that it may trigger a blood coagulation such that the bleeding may be stopped.

A similar need for stopping a blood flow may arise in a Transcatheter Aortic Valve Implantation (TAVI) during which a defective aortic valve may be replaced by a catheter-based intervention. During a TAVI, a new aortic valve may be attached to a (self-expanding) stent and delivered to the heart of a patient by inserting the catheter into the Arteria femoralis of the patient and a subsequent transfemoral movement of the catheter into the heart of the patient. Such a minimally invasive surgery may significantly reduce the risk for a patient as it may arise from a conventional replacement of an aortic valve which may require an opening of the chest of the patient. However, an undesired side-effect of TAVIs may be the formation of paravalvular leakages. Such paravalvular leakages may be understood as gaps in between an implanted aortic valve and an inner wall of a surrounding vessel (i.e., the aorta) of the patient, wherein the paravalvular leakage may allow blood flowing from the aorta back into the ventricle. This may lead to a physiological performance loss of the patient and/or breathlessness. Paravalvular leakages may at least in part be compensated by winding a sponge-like and/or swellable material (e.g., as a sealing element similar to an O-ring) around the stent and the new aortic valve such that the swellable material may at least in part close the paravalvular leakage.

These known materials are typically based on bacterial nano-cellulose, a decellularized tissue matrix (e.g., pericardium, colon or rumen) as well as on collagen-based sponges. However, these materials exhibit a variety of drawbacks. Notably, bacterial cellulose is a comparably new material for which only limited clinical long-term data is available with a view to the tolerability and durability in medical applications. Collagen-based sponges are preferably used for applications in dentistry. However, since they are often used as a native material (and are e.g., resorbable), their potential spectrum of applications is strongly limited. In particular, due to their resorbable properties, they are inherently not suitable for applications in which a long-term usage of the collagen-based sponge is required. This essentially excludes collagen-based sponges from using them for the treatment of, e.g., paravalvular leakages in which a collagen-based sponge may be required to remain in the heart of the patient for, e.g., several years. A sponge is a porous material having pores.

The preparation of the mentioned materials typically involves a drying step (e.g. reduction of water content in the biological material to below 10 wt. %), e.g., by freeze-drying. However, as a result of the drying process, these materials often exhibit a limited flexibility or even lose their flexibility which renders their handling uncomfortable or even impossible for some applications. As an example, conventionally dried materials may not be foldable, bendable to such an extent which may be required by some medical applications (e.g., in combination with a stent).

Moreover, conventional drying procedures in air often cause irreversible damages in the material and/or may lead to a certain, undesired brittleness of the material. These drawbacks may additionally be accompanied by an undesired change of the shape of the material when being dried. These disadvantageous effects may remain even after exposing the material to, e.g., blood in a medical application such that they may not be able to fulfill their task anymore for which they had originally been designed.

In other manufacturing methods of the biological material may undergo a more advanced “stabilized drying” process during which one or more chemical stabilizers may be integrated in sequential rinsing processes. However, due to the typically sponge-like texture of the materials, such a sole rinsing process oftentimes only provides a limited stabilization of the material, as the stabilizer (e.g., a stabilization fluid) may only enter the sponge-like material in a limited manner due to the typically porous structure of the material. In other words, the stabilization fluid may only access areas of the material which lie on the outside of the material whereas the stabilization fluid may not penetrate to a center portion of the material. As a result, a sufficient stabilization of the material may not be ensured and the required properties of the material, in particular, with respect to flexibility without causing irreversible structural damage cannot fully be achieved.

Hence, biological materials for medical applications known in the art and the associated methods for manufacturing and preparing them for the medical application, cannot always achieve the desired requirements for medical applications in a satisfying manner.

### SUMMARY OF THE INVENTION

A preferred method for preparing a porous biological material for a medical application includes exposing the biological material to a first liquid, repeatedly applying a compression force onto the biological material over a plurality of compression intervals, and drying the biological material The method preferably includes exposing the biological material to a second liquid after a first compression of the repeatedly compressing, wherein the second liquid includes at least 10% of a component other than water, and then applying a second compression of the repeatedly compressing. The repeatedly applying preferably includes alternating compression intervals and at least one relaxation interval between ones of the compression intervals.

### BRIEF DESCRIPTION OF THE DRAWINGS

The following figures are provided to support the understanding of the present invention:

FIGS. 1A-1B: Illustration of an exemplary air-dried piece of pericardial material before and after rehydration;

FIGS. 2A-2C: Illustration of exemplary molds for a solubilized biological material as used for ice templating;

FIGS. 3A-3B: Exemplary box-plot diagrams, illustrating the reduction of thickness and surface area of a biological material after different processing steps;

FIGS. 4A-4D: Exemplary images of a biological material taken with a scanning electron microscope;

FIG. 5: Exemplary comparison of different samples of biological material exposed to different combinations of stabilizing solution;

FIG. 6: Exemplary process diagram for manufacturing a biological material for being used in a medical application.

### DETAILED DESCRIPTION OF THE PREFERRED EMBODIMENTS

A first aspect relates to a method for preparing a biological material for a medical application. The method may include exposing the biological material to a first liquid, wherein the first liquid may for example include, by, e.g., at least 10%, a component other than water. Moreover, the method may include applying a first compression step onto the biological material, wherein the first compression step may include repeatedly applying a compression force in a plurality of compression intervals. The method may further include drying the biological material, e.g. at least in part after the first compression step.

The first liquid may include by, e.g., at least 10%, a component other than water by weight. Alternatively, the first liquid may include by, e.g., at least 10%, a component other than water by volume. In some embodiments, the first liquid may include by, e.g., at least 20%, 30%, 40%, 50%, 60%, or 70%, a component other than water (by weight or volume).

In a preferred embodiment, the first liquid may include a stabilization solution and/or a rinsing fluid. For example, the first liquid may include as stabilizer, such as polyethylene glycol (PEG), such as PEG-200 or PEG-400, and/or glycerin, by at least 10%.

The compression force may be applied to the biological material in at least two compression intervals, preferably at least three, four or five compression intervals. The compression force may be applied to the biological material such that it at least temporarily reduces a thickness of the biological material.

The aforementioned method may provide the advantage of providing a dried biological material for medical applications with reduced dimensions and which may be stored in a dry environment (e.g., in the absence of preserving liquids). Moreover, the biological material may be provided with swelling properties upon an exposure to a liquid, e.g., blood. These swelling properties may allow that the biological material, with reduced volume, may be delivered to a certain position in the body of a patient, e.g., by a catheter-based delivery, at which the biological material may increase its volume due to the contact with blood. This may allow a space-saving delivery of the biological material. Moreover, due to the expansion of the biological material upon a contact with, e.g., blood, the biological material may be expandable into small cavities (e.g., into a wound/lesion) of the body of the patient at which the biological material may contribute to a blood coagulation such that an undesired bleeding and/or a blood flow may be stopped.

Moreover, besides the aforementioned advantages, the method may allow providing a biological material with improved long-term durability such that the biological material may in particular be suitable for being used in the reduction of undesired side-effects in TAVI, e.g., for the at least partial compensation of paravalvular leakages for which the biological material may remain in the body of the patient (e.g., for several years). The biological material may generally be applied, e.g., in any kind of surgery which preferably requires a flow of blood or any other body liquid to be reduced and/or stopped.

Another advantage of the biological material may intrinsically be seen in an increased biocompatibility, as no synthetic materials may be used, which may contribute to a risk reduction for the emergence of thromboembolic complications.

The repeated application of a compression force after or while the biological material is exposed to the first liquid may increase the penetration of the biological material by the first liquid. For example, if the first liquid includes a rinsing fluid, this may lead to an improved displacing, e.g. of previously applied cross-linking agents (such as glutaraldehyde. Also, if the first liquid includes a stabilizer, the penetration of the biological material by the stabilizer may be improved, and more water may be displaced out of the material, such that the drying is improved. In some example, the formation of hydrogen bridges during drying that may lead to bonds that are irreversible (even upon rehydration) may thus be reduced.

The repeatedly applying of the compression force may include alternating compression intervals and at least one relaxation interval of the biological material.

In some preferred embodiments, a compression interval may be followed by at least one relaxation interval, wherein, during the at least one relaxation interval, the compressed biological material is allowed to return to its state prior to the application of the compression force such that, e.g., a reduction in thickness of the biological material (e.g., as a result of the application of the compression force) may at least partially be reversed.

The compression force may be configured to be equal in magnitude during each of the alternating compression intervals. Alternatively, it may also be possible that the compression force is decreased with each subsequent compression interval. Alternatively, it may also be possible that the compression force is increased between each subsequent compression interval.

By applying the compression force in alternating compression intervals and at least one relaxation interval, an improved saturation/impregnation of the biological material with (at least) the first liquid may be ensured. More specifically, during each compression interval, the first liquid (and/or other liquids) residing in the biological material may be displaced from the biological material whereas in a relaxation interval, the first liquid may be soaked into the biological material (again). A repetition of said procedure may lead to an increased amount of the first liquid in the biological material and a penetration depth of the first liquid into the biological material may be increased. This may contribute to a saturation of the biological material with the first liquid. If the first liquid is a stabilizing liquid, an improved stabilization process may thus be supported.

The at least one relaxation interval may include 0.2 s to 30 s, preferably 0.5 s to 15 s. In a preferred embodiment, the at least one relaxation interval may be provided with a duration of about 3 s to 7 s, e.g. about 5 s.

If the biological material is exposed to at least two relaxation intervals, the at least two relaxation intervals may be of equal duration. Alternatively, it may be possible that the duration of a relaxation interval is increased or decreased with each subsequent relaxation interval.

By adapting the at least one relaxation interval such that it includes 0.2 s to 30 s, an optimized impregnation of the biological material with the first liquid may be ensured as shorter relaxation intervals may not ensure that the biological material is provided with a sufficient amount of time to soak a sufficient amount of the first liquid which may then allow a desired extent of, e.g., stabilizing of the biological material (if the first liquid is a stabilizing solution). On the other hand, if the relaxation interval is chosen longer than the aforementioned interval, the manufacturing of the biological material for a medical application may be unduly extended in time.

The compression intervals may include 0.1 s to 10 s, preferably 0.2 to 5 s. In a preferred embodiment, the at least one compression interval may be provided with a duration of about 1 s to 3 s, for example about 2 s.

If the biological material is exposed to at least two compression intervals, the at least two compression intervals may be of equal duration. Alternatively, it may be possible that the duration of a compression interval is increased or decreased between each subsequent compression interval.

By adapting the compression interval such that it includes 0.1 s to 30 s, an optimized distribution of the first liquid within the biological material may be ensured and ejection of undesired liquids within the biological material are removed. However, if the duration of the compression interval was chosen longer than the aforementioned interval, the manufacturing of the biological material may be unduly extended in time.

The first compression step may include applying a compression force in a range of 25 kPa to 500 kPa, preferably 50 kPa to 450 kPa. In a preferred embodiment the compression force may be 70 kPa to 80 kPa, e.g. about 74 kPa, or 350 kPa to 400 kPa, e.g. about 370 kPa. These values have for example turned out to be very suitable for biological material including pericardium.

The compression force may be applied to the biological material across its entire main surfaces. For example, if the biological material is provided as a sheet like material, the compression force may be applied perpendicularly to the sheet's front and back sides.

Configuring the first compression step such that the compression force lies in the aforementioned range may ensure a reproducible compression of the biological material to a certain, desired thickness. As the applicant has found, higher compression forces may lead to a scattering of the obtained values of the thickness of the biological material (after finishing the method) which may thus not lead to properties of the biological material in reproducible manner (e.g., with respect to the thickness of the biological material and/or its volume). Moreover, higher compression forces may also damage the biological material whereas a compression force which is too weak to compress the biological material may not lead to the aforementioned exchange of the first liquid in the biological material and may thus not lead to the desired extent of cross-linking or stabilizing in the biological material.

The method may further include exposing the biological material to a second liquid including, e.g., by at least 10%, a component other than water. The exposing to the second liquid may be carried out after exposure to the first liquid.

The method may further include applying a second compression step onto the biological material. The second compression step may be carried out, for example, during the exposition of the biological material to the second liquid. The second compression step may be similar to the first compression step and include features described herein with reference to the first compression step, for example.

The second liquid may include a different composition as compared to the first liquid. The second liquid may include by, e.g., at least 10%, a component other than water by weight. Alternatively, the second liquid may include by, e.g., at least 10%, a component other than water by volume. In some embodiments, the second liquid may include by, e.g., at least 20%, 30%, 40%, 50%, 60%, or at least 70% a component other than water (by weight or volume).

The second liquid may include a rinsing solution and/or a stabilizer, for example. If the second liquid includes a stabilizer, it may preferably include polyethylene glycol (PEG), such as PEG-200 or PEG-400, and/or glycerin.

The application of at least two stabilizer solutions (e.g., if the first liquid is a first stabilizer solution and if the second liquid is a second stabilizer solution) may enhance the stabilization of the biological material. In this example, the first and second pressing steps may be carried out during exposure of the biological material to the first and second stabilizer, respectively. In other examples, the first liquid may include or be a rinsing solution, whereas the second liquid includes or is a stabilizer.

The method may further include exposing the biological material, after the second compression step, to a third liquid including, by at least 10%, a component other than water. The method may further include applying a third compression step onto the biological material. The third compression step may be similar to the first compression step and include those features described herein with reference to the first compression step, for example.

The third liquid may include a different composition as compared to the first liquid. The third liquid may include a different composition as compared to the second liquid. The third liquid may include by, e.g., at least 10%, a component other than water by weight. Alternatively, the third liquid may include by, e.g., at least 10%, a component other than water by volume. In some embodiments, the third liquid may include by, e.g., at least 20%, 30%, 40%, 50%, or at least 60% a component other than water (by weight or volume).

The third liquid may be a −a stabilizer. The third liquid may preferably include polyethylene glycol (PEG), more preferably PEG-400 and/or PEG-200 and/or glycerin.

In some embodiments, the method may further include exposing the biological material to a fourth, a fifth, a sixth, a seventh, etc. liquid. The exposing of the biological material to the fourth, the fifth, the sixth, the seventh liquid, etc. may be accompanied by applying a respective fourth, fifth, sixth, seventh, etc. compression step onto the biological material.

The respective exposing times may be similar or equal to the exposing times described herein with reference to the first liquid, the second liquid and the third liquid, above.

The drying may include drying the biological material on a material including the first, second and/or third liquid. The drying may be performed in a climate cabinet and/or in a desiccator.

The drying may be adapted such that it may predominantly reduce the amount of water and/or (an excess of remnant) stabilizer (e.g., of the first liquid, the second liquid, the third liquid, etc.) in the biological material.

The drying process may further include applying a compression force onto the biological material. The compression force may be applied to the biological material during the entire drying process or only for at least one portion of the drying process or after the drying process.

The drying process may be performed at room temperature (e.g., at about 20° C.). Alternatively, it may also be possible that the drying process is performed above room temperature (e.g., at 30° C., 40° C., 50° C., 60° C., 80° C., etc.). Alternatively, it may also be possible that the drying process is performed below room temperature (e.g., at 18° C.). The drying process may preferably occur at a constant temperature. However, in some embodiments a variation of the temperature may be advantageous.

The material impregnated with the liquid may act as a reservoir, adapted to provide the biological material with the liquid during the drying process. The material impregnated with the liquid may be a sheet- or towel-like element. Alternatively, the material impregnated with the liquid may be a grid-like structure which has been covered with the liquid prior to placing the biological material thereon.

In some embodiments, it may also be possible that the material is impregnated with a fourth liquid, etc.

By performing the drying process in a material impregnated with the first, second or third liquid, it may be avoided that the drying process extracts undue amounts of the respective liquid (e.g. stabilizer) from the biological material. Stabilizer extracted from the biological material (due to the drying process) may be replaced by stabilizer from the impregnated material such that a certain amount of stabilizer may be maintained in the biological material. Therefore, an undesired deterioration of the biological material during the drying process may be avoided.

The method may cause a reduction of a thickness of the biological material by 40-80% or 50-70% as compared to an initial thickness of the biological material. The reduction of the thickness may preferably be approx. 60%.

The method may cause a reduction of the thickness of the biological material by 50-70% after compression of the biological material, e.g. in a climate cabinet, wherein the reduced thickness is maintained in this range for at least for 1-7 days (e.g., after 1 d, 2 d, 3 d, 4 d, 5 d, 6 d or 7 d).

Additionally or alternatively, the method may also cause a reduction in surface area of the biological material by 10-30%, preferably by approx. 15%. The surface area may be associated with the metric dimensioning (e.g., width and length) of the area which experiences the compression force as described above, e.g. in case the biological material is provided in a sheet like manner.

By reducing the thickness (and the surface area) of the biological material, a compact biological material for a medical application may be provided. This may in particular allow a delivery and/or insertion of the biological material into narrow vessels and/or lesions in the body of the patient.

The first, second and/or third liquid may include a stabilizer such as glycerin and/or polyethylene glycol, PEG, by at least 10%. The first liquid may alternatively include a rinsing solution.

In exemplary embodiments, in which the first, second and/or third liquid includes glycerin, the glycerin may be provided with a concentration of 10-50%, preferably with a concentration of 20-30%.

In exemplary embodiments, in which the first, second and/or third liquid includes a polyethylene glycol, the PEG may be provided with a concentration of 10-50%, preferably with a concentration of 20-40%.

By providing the first liquid such that it includes glycerin and/or PEG, preferably with the aforementioned concentrations, an optimized stabilization of the biological material may be obtained.

In some embodiments, wherein the first or second liquid includes a rinsing solution, also saline solution may generally be used that may include less than 10% of a component other than water (e.g. the NaCl may be provided as a 0.9% solution). Also, ultrapure water may be used, for example. Hence, it is also a separate aspect that one or more (pulsatile) compression steps, as described herein, are applied to the biological material, before drying the biological material, e.g. during exposure to a liquid.

The biological material may include a pericardial tissue. The pericardial tissue may preferably be a porcine pericardium. Alternatively or additionally, the biological material may additionally or alternatively include other biological materials which may preferably include collagen fibers. By providing the biological material as a pericardial tissue, the biocompatibility of the biological material in the body of the patient may be increased.

Moreover, by using porcine pericardial tissue, the acquisition costs of the biological material may be reduced and a cost efficient biological material for a medical application may be provided. In other embodiments, the biological material may include a colon or rumen tissue, for example.

In a preferred embodiment, the biological material may (initially) include a native (i.e., natural) biological material. Additionally, the biological material may include a decellularized biological tissue.

Another aspect relates to a biological material which may be prepared according to one of the aforementioned method steps. By providing a material, manufactured according to one of the aforementioned method steps, a storable (in a dry environment), sponge-like and expandable/swellable material for medical applications may be provided that is highly swellable.

The biological material may be expandable to at least 70%, preferably at least 80%, e.g. 80-90%, of its initial thickness by rehydration, e.g. upon a contact with an aqueous liquid. Additionally or alternatively, the biological material may be expandable to at least 70%, preferably at least 80% of its initial surface area by rehydration, e.g. upon a contact with an aqueous liquid.

In a preferred embodiment, the biological material, prepared according to one of the aforementioned method steps, may be provided in a reduced size as compared to the initial dimensions of the biological material.

The biological material may be expandable/swellable as a result of rehydrating the biological material. The rehydration may be caused upon exposing the biological material to a liquid, preferably blood.

The biological material may be adapted such that the expansion occurs within a time interval of less than 30 s, preferably of less than 20 s, more preferably of less than 10 s.

By providing the biological material such that it is expandable in thickness and/or its areal dimensions, an expansion of the biological material into small cavities may be supported such that an undesired bleeding may be stopped within a short amount of time.

Another aspect relates to an implant. The implant may include a biological material prepared according to one of the aforementioned method steps.

The implant may be a stent, preferably a self-expandable stent. The stent may include an aortic valve.

The implant may be provided with the biological material such that the biological material surrounds the implant along a circumferential direction and such that upon an expansion (e.g. in a radial direction) of the biological material (e.g., in response to a rehydration), a blood flow between the biological material and an inner wall of a blood vessel of the patient may be prevented.

Another aspect relates to a use of a material, which may be prepared according to one of the aforementioned method steps, for a sealing element of an implant.

By providing an implant with the biological material prepared according to one of the aforementioned method steps, the risk for undesired side effects, e.g., paravalvular leakages, as they may occur during the implantation of, e.g., an aortic valve, may be suppressed as a potentially arising paravalvular leakage may immediately be treated by the expanding biological material. The latter step may contribute to at least contribute to a decrease of a paravalvular leakage.

With respect to the foregoing entire disclosure, the present invention further includes the following embodiments:

A method for preparing a porous tissue sponge, wherein the method includes the steps of:
providing one or more native biological tissue(s) or one or more decellularized biological tissue(s) containing collagen fibers, preferable pericardial tissue or decellularized pericardial tissue,
(dry) mechanical solubilization of the one or more native biological tissue(s) or one or more decellularized biological tissue(s), preferably by grinding, to obtain a solubilized tissue,
homogenization of the solubilized tissue to obtain a homogenized tissue,
pouring the homogenized tissue in a frozen aldehyde mold, preferably a frozen glutaraldehyde mold, and freezing the homogenized mixture to obtain a frozen tissue,
defrosting the frozen tissue and the frozen aldehyde mold, preferably a glutaraldehyde mold, thereby pre-cross-linking the collagen fibers of the tissue to obtain a porous tissue sponge,
optionally further cross-linking the porous tissue sponge by adding an aldehyde, preferably a glutaraldehyde, at elevated temperature e.g. 60 degrees.

A Method (600) for preparing a biological material for a medical application, including:
providing a porous tissue sponge, preferably obtained by the above method of
exposing the porous tissue sponge to a glycerin solution;
applying a first compression step onto the biological material, wherein the first compression step includes repeatedly applying a compression force in a plurality of compression intervals; and
exposing the porous tissue sponge to an aqueous polyethylene glycol solution, including polyethylene glycol having an average molecular weight of between 150 g/mol and 300 g/mol;
applying a second compression step onto the biological material, wherein the second compression step includes repeatedly applying a compression force in a plurality of compression intervals; and
exposing the porous tissue sponge to an aqueous polyethylene glycol solution, including polyethylene glycol having an average molecular weight of between 300 g/mol and 1000 g/mol;
applying a third compression step onto the biological material, wherein the third compression step includes repeatedly applying a compression force in a plurality of compression intervals to obtain a stabilized tissue sponge; and
drying the stabilized porous tissue sponge to obtain a dried sponge; and
optionally compressing the dried sponge.

The repeated application of the compression force preferably includes alternating compression intervals and at least one relaxation interval of the biological material.

The at least one relaxation interval can be 0.2 s to 30 s, preferably 0.5 s to 15 s.

The compression intervals can be 0.1 s to 10 s, preferably 0.2 to 5 s.

The first compression step can include applying a compression force in a range of 25 kPa to 500 kPa, preferably 50 kPa to 450 kPa.

The drying is preferably done by the reduction of the relative humidity to 10% or less at 30 to 40° C., preferably 37° C.

The compressions can be conducted to cause a reduction of a thickness of the biological material by 40-80%, preferably by 50-70%, as compared to an initial thickness of the biological material.

The porous tissue sponge is based on pericardial tissue.

The dried sponge is preferably compressed by applying a compression force of 50 kPa to 300 kPa.

The dried sponge is preferably compressed by applying a compression force of 70 kPa to 80 kPa.

The biological material can be expandable to at least 70%, preferably at least 80% of its initial thickness by re-hydration; and/or the biological material is expandable to at least 70%, preferably at least 80% of its initial surface area by re-hydration.

The biological material can be applied to an implant, as a sealing element.

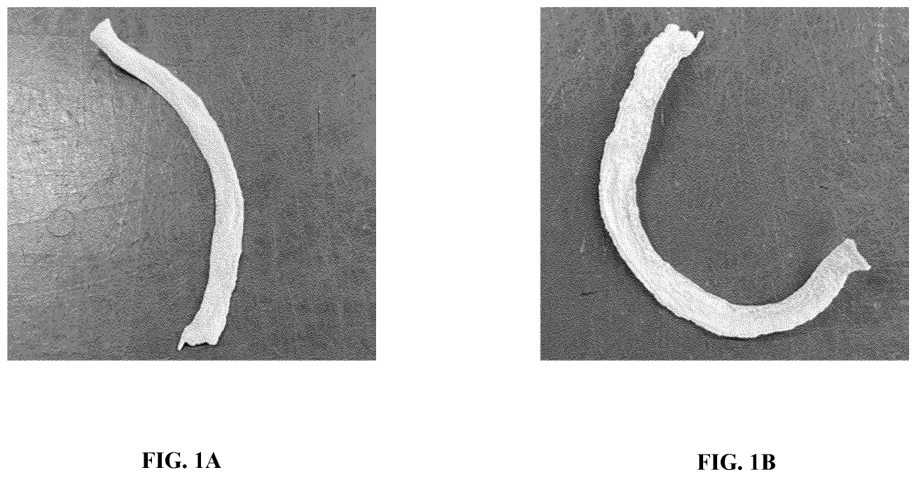

FIGS. 1A and 1B exemplary show a sponge-like piece processed from porcine pericardial tissue which has conventionally been dried in air. More specifically, FIG. 1A shows the air-dried pericardial tissue in a dried state. The piece of pericardial tissue had initially been provided as a sheet like material, with a rectangular shaped surface. As depicted in FIG. 1A, the pericardial tissue acquired a bended and uneven shape and lost its rectangular shaped surface as a result of the air-drying procedure and as a result of the associated loss of water. In particular, the extraction of water from the pericardial tissue caused the pericardial tissue to become brittle and inflexible (i.e., not deformable) which is associated with an irreversible damage of the internal structure of the pericardial tissue.

FIG. 1B shows the pericardial tissue of FIG. 1A in a state in which the pericardial tissue has been rehydrated (e.g., exposed to a liquid, e.g., water). As depicted in FIG. 1B the pericardial tissue acquired an even more degenerated and deteriorated outer shaped after the rehydration as compared to the pericardial tissue shown in FIG. 1A.

From the results depicted in FIGS. 1A and 1B, it can be concluded that exposing a piece of pericardial tissue to a conventional drying procedure irreversibly damages the pericardial tissue and does not lead to the desired properties of the pericardial tissues which are required for medical applications (such as, e.g., flexibility, etc.).

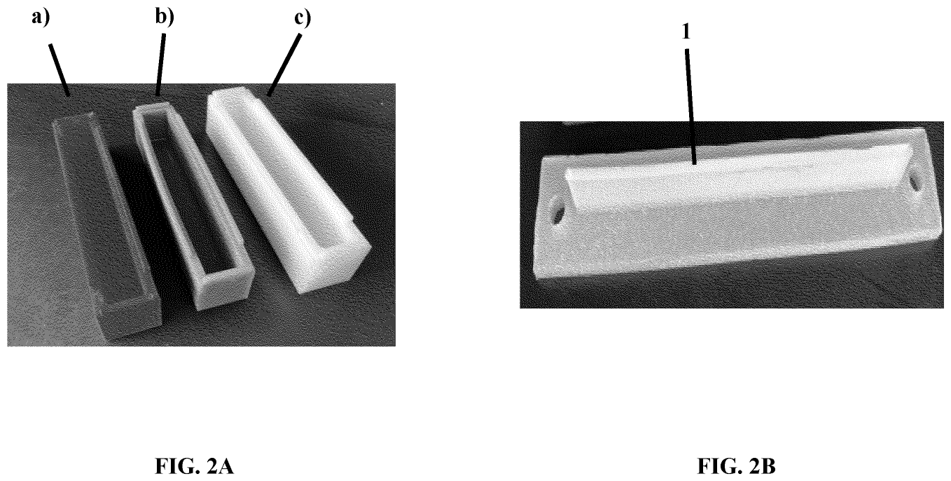

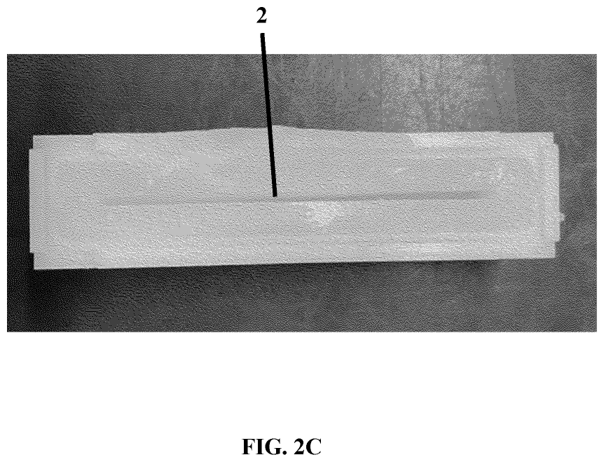

FIGS. 2A-2C show different tools, which may be used to transform an initial biological material, preferably a pericardial tissue, into a (sponge-like) material of desired shape.

Notably, FIG. 2A shows three different molds made from different materials which may be used to form a mold, e.g. made from glutaraldehyde, for shaping a solubilized biological material.

With reference to FIG. 2A, mold 100 is made from polylactic acid (PLA), mold 200 is made by selective laser sintering (SLA) and mold 300 is made from polyoxymethylene (POM). As depicted in FIG. 2A, each of the exemplary molds 100, 200, 300 is provided with a cuboid-shaped inner volume.

FIG. 2B shows an exemplary lid adapted to close a mold 100, 200, 300 as shown in FIG. 2A. The exemplary lid may preferably be made from silicone and may be provided with a protruding element 1 having a rectangular cross-section.

According to an aspect of the invention, one of the exemplary molds, as depicted in FIG. 2A, may be filled with a glutaraldehyde solution (preferably with a concentration of about 0.1 to 5, e.g. about 0.5%) and covered by the exemplary lid as depicted in FIG. 2B. The lid of FIG. 2B may cover the mold such that the protruding element 1 enters the glutaraldehyde solution. The mold filled with glutaraldehyde solution may then be frozen.

FIG. 2C exemplarily shows a frozen block of glutaraldehyde after the glutaraldehyde solution has been frozen in a mold of FIG. 2A. After the freezing process, the lid may be taken off from the respective mold and at the location at which the protruding element 1 was in contact with the glutaraldehyde during the freezing process, a rectangular recess 2 was formed in the frozen block of glutaraldehyde. The block of FIG. 2C may act as a mold (also referred to herein as template).

In a subsequent step, an initial biological material (preferably a porcine pericardium) may be solubilized (as it will further be described below). The solubilized biological material may then be filled into the rectangular recess 2 of the template (e.g., by a syringe while avoiding the formation of air bubbles in the solubilized biological material) and may be frozen at a temperature of at most −20° C. During the freezing process, ice crystals may generally be formed in the solubilized biological material which may lead to the formation of a sponge-like/porous structure in the biological material (e.g. such that the material may be compressible at room temperature, liquid may enter the pores, etc., after defrosting).

After the initially solubilized biological material has been fully frozen, it may (slowly) be defrosted at room temperature. During the defrosting process, the biological material may be cross-linked by the melting glutaraldehyde and at least partially be stabilized. Said effect of stabilization may additionally be enhanced during subsequent stabilization steps as described in further detail, below.

It is noted that the shape of the initial mold (as exemplarily depicted in FIG. 2A) and the shape of the lid (as depicted in FIG. 2B) may generally be provided in any possible geometry. As a result, the such obtained glutaraldehyde-template may also be provided in any possible shape. Such an ice templating procedure may thus facilitate that a biological material may be provided in a variety of different shapes such that the biological material may be applicable for a wide range of different applications. This process is also referred to as ice templating.

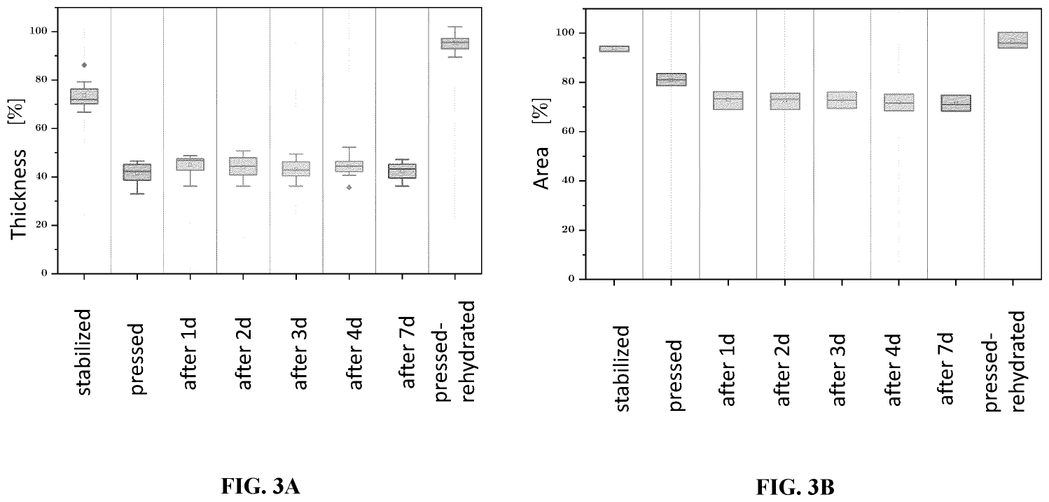

FIGS. 3A and 3B show exemplary box-plot diagrams indicating the reduction in thickness (FIG. 3A) and surface area (FIG. 3B) at different stages of the manufacturing process of the biological material as measured in a test series.

FIG. 3A shows that after applying stabilization related steps to the biological material (e.g., after exposing the biological material to the first liquid, the second liquid and the third liquid and the associated compression steps as described herein) the biological material obtains a reduced thickness.

FIG. 3A further shows that as a result of stabilization, the thickness of the biological material may decrease from 100% to approx. 60-90%, or 65-80% of the initial thickness of the biological material. An additional compression (pressing) of the biological material, preferably at 74 kPa at room temperature, e.g. after drying as described herein (see, e.g., exemplary process step 615 as described with reference to FIG. 6, below), may lead to a further reduction of the thickness of the biological material to approx. 30-55% or 35-50% of the initial thickness of the biological material.

A subsequent storing of the biological material for several days (data for 1, 2, 3, 4, 7 days is shown) in a desiccator does not lead to a significant change of the thickness of the biological material as compared to its initial thickness. Hence, the material can be prepared in a stable state.

FIG. 3A further depicts that a rehydration of the pressed biological material may lead to a recovery of the thickness up to 100% of the original thickness of the biological material.

FIG. 3B shows the reduction in surface area in dependence on the processing steps of the biological material as described with reference to FIG. 3A. It is noted that a reduction of the surface area, besides a reduction of the thickness of the biological material, is generally seen as advantageous as it leads to a volume reduction of the biological material. This may in particular allow the application of the biological material in minimally invasive applications for which only small pieces of biological material may be used.

Similar to the reduction in thickness, the stabilization process also leads to a reduction in surface area in the range of 85-99%, e.g. approx. 90-95% of the original surface area of the biological material. An additional compression (pressing) of the biological material, preferably at 74 kPa at room temperature, and preferably after drying, e.g. as described herein, may lead to a further reduction of the surface area of the biological material to approx. 75-85% of the initial surface area of the biological material. This result may be based on the aspect that the biological material may preferably be placed on a dedicated filter paper which, during the compression of the biological material, may absorb (excess) stabilizer solution leaking from the biological material as a result of the applied compression force. As a result, the biological material may also undergo a shrinkage with respect to the surface area of the biological material.

A subsequent storing of the biological material in a desiccator shows a slight further decrease of the surface area to about 65-80%. Further storage for several days in a desiccator (data for 2, 3, 4, 7 days is shown) does not lead to a significant further change of the surface area of the biological material as compared to its initial surface area. Hence, the material can be prepared in a stable state.

FIG. 3B further depicts that a rehydration of the pressed biological material may lead to a recovery of the surface area up to 100% of the original surface area of the biological material.

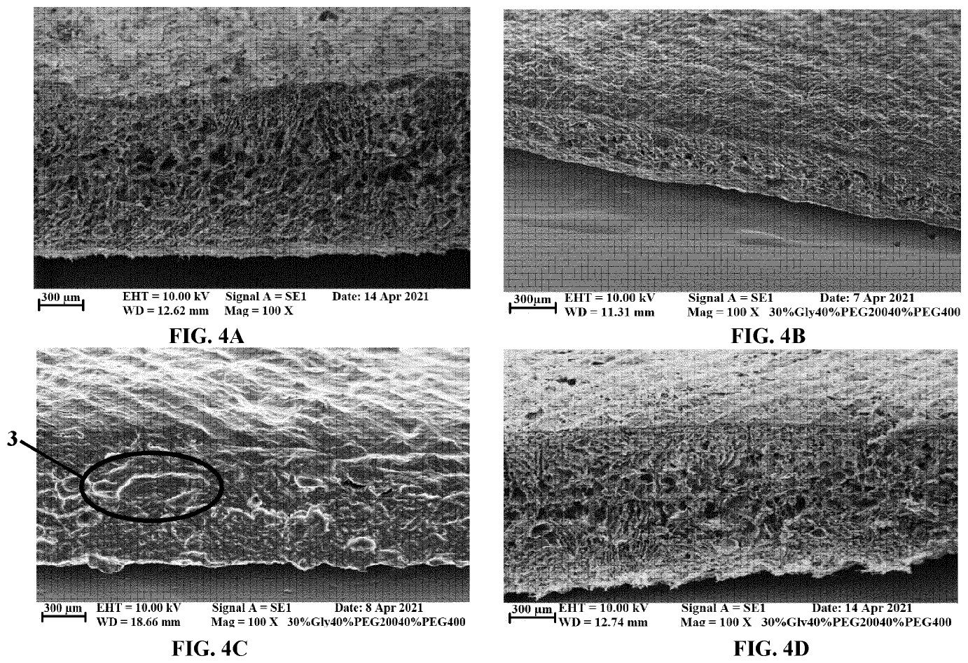

FIGS. 4A-4D show images of the biological material (porcine pericardium), taken with a scanning electron microscope, after having exposed the biological material to different processing steps as outlined herein. It is emphasized that FIGS. 4A-4D were taken at the same scale.

FIG. 4A shows a cross-sectional view of the biological material after cross-linking and rinsing, but prior to the application of a stabilizer (e.g. in a state as obtainable after step 609 outlined with reference to FIG. 6). In particular, FIG. 4A shows the internal fibrous structure of the biological material. The thickness of the sheet-like biological material may include about 0.2 to 5 mm, for example, about 0.5 to 2 mm, or about 1 to 1.5 mm

FIG. 4B shows a cross-sectional view of the biological material after applying the stabilization, drying and final compression step applied to the biological material (e.g. in a state after step 615 as outlined with reference to FIG. 6). As can be seen in FIG. 4B, the thickness of the biological material has significantly been decreased as compared to the initial thickness of the biological material (FIG. 4A).

This can also be seen in FIG. 4C which shows an enlarged view of the sample shown in FIG. 4B. FIG. 4C shows that the fibers of the biological material have additionally been covered by a protective layer of stabilization solution as a result of applying the stabilization step. Reference numeral 3 exemplarily highlights an exemplary area showing the protective layer as a slightly blurred fiber structure.

FIG. 4D shows a cross-sectional view of the biological material after a rehydration of the (compressed) biological material as depicted in FIGS. 4B and 4C. As can be seen in FIG. 4D, the biological material returns to almost its initial thickness after the rehydration. As compared to FIGS. 4B and 4C, FIG. 4D also shows that the fibrous structure, as initially depicted in FIG. 4A, is re-developed as a result of the rehydration. Moreover, FIG. 4D also shows that no irreversible damages arise in the internal fibrous structure of the biological material due to the processing steps and the rehydration.

To summarize, FIGS. 4A-4D show the internal structure of the biological material after the application of several processing steps as outlined herein. In particular, a comparison of FIGS. 4A and 4D show that the initial properties of the biological material (FIG. 4A) may be re-obtained after a rehydration of the biological material (FIG. 4D) even after the application of the processing steps.

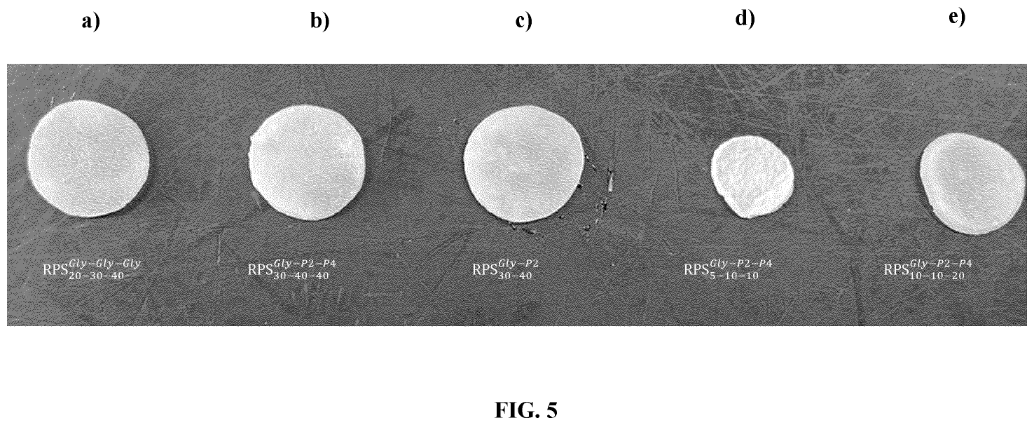

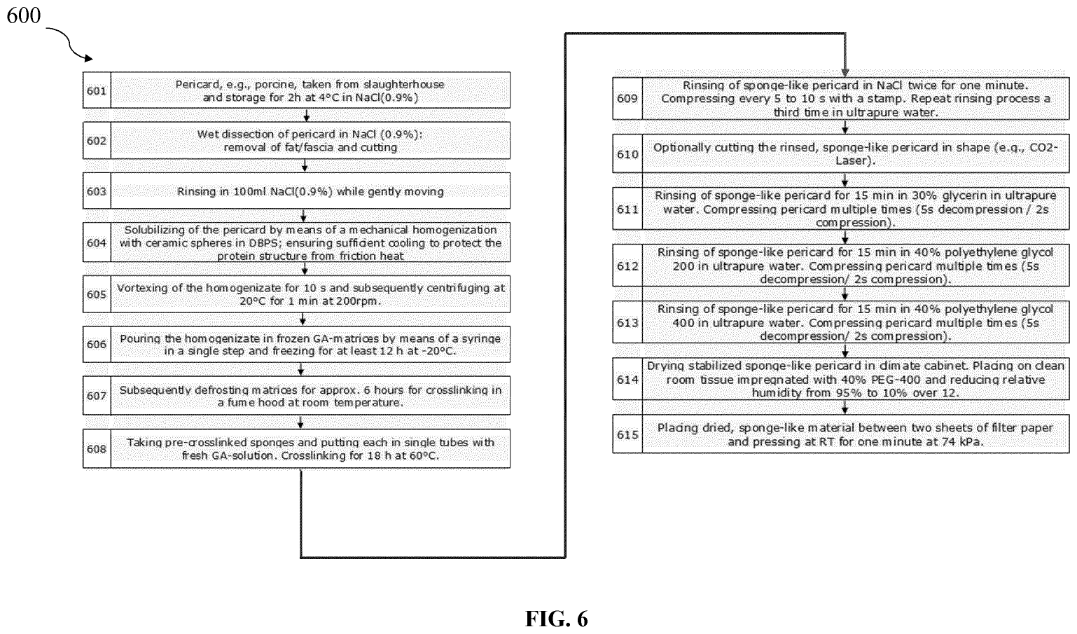

FIG. 5 shows a comparison of the effect of applying different combinations of stabilization solutions and their concentrations onto dried samples of the biological material (hereinafter referred to as re-engineered pericardium sponge (RPS). For the comparison in FIG. 5, different samples of a biological material have been prepared with initially identical dimensions. Each of the initially prepared samples has then been exposed to different combinations of stabilizing solutions. Each of the samples of biological material was exposed to each stabilization solution for 15 min. The resulting biological materials are directly compared to each other in FIG. 5 as samples a)-e).

Sample a) shows the biological material after exposing the biological material three times to glycerin with a concentration of 20% (first liquid), 30% (second liquid) and 40% (third liquid). During each exposure time, a pulsatile compression was applied as outlined herein (the same for all exposure times and samples). Sample a) depicts that the used combination of stabilization solutions leads to excellent results, i.e., the biological material exhibits flexibility and does not become brittle. Moreover, sample a) also maintains its outer shape and dimensioning. In some example, a concentration of 15-25% glycerin, 25-35% glycerin and 35-45% glycerin may be used.

Sample b) shows another sample of the biological material after an exposure to glycerin (with a concentration of 30%), PEG-200 (with a concentration of 40%) and PEG-400 (with a concentration of 40%). Sample b) also shows no loss of flexibility and sample b) did not become brittle. The combination of the stabilizing solutions applied to sample b) has been determined to be the preferred combination of stabilizing solutions. In some example, a concentration of 25-35% glycerin, 35-45% PEG-200 and 35-45% PEG-400 may be used.

Sample c) shows the biological material after an exposure to glycerin (with a concentration of 30%) and PEG-200 (with a concentration of 40%). Sample c) lacks the third stabilization step (as compared to samples a) and b)). After rehydrating sample c), the sample c) does not fully evolve an acceptable thickness (e.g., above 90% of the initial thickness of the biological material) after the rehydration anymore which may be due to the missing third stabilization step. The combination of stabilizing solutions as applied to the biological material with respect to sample c) may be good for some applications but it can be improved by the third stabilization step applied to samples a) and b).

Sample d) shows the biological material after an exposure to glycerin (with a concentration of 5%), PEG-200 (with a concentration of 10%) and PEG-400 (with a concentration of 10%). As can be seen in FIG. 5, sample d) shows a deformation, a loss of flexibility and sample d) became brittle after the application of the stabilization steps. It can thus be concluded that the combination of stabilization solutions applied to sample d) leads to unsatisfying properties of the biological material. This may arise from the reduced concentrations of stabilizing solutions used for the treatment of sample d) (as compared to sample b)).

Sample e) shows the biological material after an exposure to glycerin (with a concentration of 10%), PEG-200 (with a concentration of 10%) and PEG-400 (with a concentration of 20%). As can be seen in FIG. 5, sample e) shows a deformation, a loss of flexibility and sample e) became brittle after the stabilization steps. It can thus be concluded that the combination of stabilization solutions applied to sample e) leads to unsatisfying properties of the biological material. This may be explained as arising from the reduced concentrations of stabilizing solutions used for the treatment of sample e) (as compared to sample b)).

It can be concluded that the chosen stabilization solutions and the chosen selection of the concentrations of the stabilization solutions have a severe impact on the properties of the biological material (e.g., with respect to flexibility, rehydration properties and brittleness).

The following table further shows an overview of the surface areas obtained after stabilizing the biological material in different sequences of stabilizer solutions after the compression step, after 1 d of storing the biological material and after 2 d of storing the biological material. The table refers to the obtained surface area (in percent) relative to the initial surface area of the biological material.

TABLE 1
comparison of the surface area reduction of the biological
material depending on the applied stabilizing solutions
and the applied processing step.

| Stabilizer | Compression | 1 d | 2 d |
|---|---|---|---|
| RPS₂₀₋₃₀₋₄₀^{Gly-Gly-Gly} | 81.07% | 73.34% | 73.17% |
| RPS₃₀₋₄₀₋₄₀^{Gly-PEG200-PEG400} | 93.22% | 90.47% | 90.00% |

As depicted in the table above, the exposing of the biological material to glycerin for three times leads to the most pronounced decrease of surface area of the biological material.

FIG. 6 shows an exemplary process diagram of the manufacturing process 600 of an initial piece of biological material and its preparation for being used for a medical application.

The manufacturing process may start with step 601, wherein a biological material (e.g., a pericardial material) may be obtained from a slaughterhouse. The pericardial material may preferably be stored for 2 h at a temperature of 1-5° C., preferably at 4° C. in a saline solution, e.g., in a NaCl solution with a concentration of 0.5-1.5%, preferably 0.9%.

In step 602, the pericardial material may be dissected in a wet NaCl environment (with a concentration of 0.5-1.5%, preferably 0.9%). The dissection may include the removal of fat and/or fascia from the pericardium. Step 602 may further include a (coarse) cutting of the pericardial material into a desired geometry.

In step 603, the pericardial material may be rinsed in 100 ml NaCl (with a concentration of 0.5-1.5%, preferably 0.9%) while gently moving the pericardial material.

In step 604, the pericardial material may be solubilized including a homogenization step which may be based at least in part on a mechanical interaction of the pericardial material and a plurality of ceramic spheres while being exposed to Dulbecco's Phosphate Buffered Saline (DPBS). The mechanical interaction of the plurality of ceramic spheres may be understood as a grounding of the biological material. It is further crucial for step 604 that sufficient cooling is provided to avoid any harmful effects on the protein structure of the pericardial material which may arise from frictional heat caused by the mechanical interaction of the ceramic spheres due to friction.

In step 605, the homogenized pericardial material obtained in step 604, may be vortexed for 10 s and may subsequently be centrifuged at 100-500 rpm, preferably at 200 rpm, for 1-5 min, preferably for 1 min at 15-30° C., preferably at 20° C.

The homogenized pericardial material may in step 606 be filled into a syringe and may be filled into a prepared glutaraldehyde template/matrix (as described above) and may subsequently be frozen for at least 10-15 h, preferably for at least 12 h at −15-30° C., preferably at −20° C. Due to the formation of ice crystals, the pericardial material may acquire sponge-like properties.

In step 607, the template/matrix by be defrosted at room temperature, preferably in a fume hood, for at least 5-10 h, preferably for 6 h. The defrosting is accompanied by a cross-linking of the pericardial material.

In step 608, the pre-cross-linked pericardial material may be filled into tubes with glutaraldehyde solution. The pericardial material may then be further cross-linked for 15-25 h, preferably for 18 h at 50-70° C., preferably for 60° C. For example, a pulsatile compression step as described herein may be applied to improve/accelerate the cross-linking.

In step 609, the sponge-like pericardial material may at least twice be rinsed in NaCl solution for 30-90 s, preferably for 1 min. During rinsing, a pulsatile compression step as described herein may be applied to improve/accelerate rinsing. For example, during the rinsing process, the pericardial material may be compressed with a stamp every 1-20 s, preferably every 5-10 s. The rinsing process may additionally be repeated for a third time using ultrapure water.

In step 610, the rinsed pericardial material may be cut into the final desired shape, e.g., by using a CO₂ laser.

It is emphasized that it is beneficial to repeatedly compress the pericardial material when rinsing as otherwise, it may not be ensured that the glutaraldehyde solution is fully removed from the biological material. In this regard, the applicant has investigated the amount of remnant glutaraldehyde solution in the biological material after a conventional rinsing procedure as compared to the application of a pulsatile compression of the biological material as suggested for step 609. The remnant amount of glutaraldehyde is shown in the table below for two different initial thicknesses of the pericardial material.

TABLE 2
comparison of remaining glutaraldehyde in a sample piece
of biological material depending on the applied rinsing
procedure and the thickness of the biological material.

| | | Remaining |
|---|---|---|
| Sponge-like | | glutaraldehyde/ |
| pericardium | Rinsing process | (mg/l) |
| Thickness: 2 mm | Conventional | 233.33 ± 47.26 |
| | Rinsing | |
| | Pulsatile | 21.33 ± 0.58 |
| | Compression | |
| Thickness: 5 mm | Conventional | 543.33 ± 187.71 |
| | Rinsing | |
| | Pulsatile | 39.00 ± 1.00 |
| | Compression | |

With regard to Tab. 2, the “conventional rinsing” relates to rinsing the pericardial material three times for 60 s in isotonic NaCl solution while gently moving the pericardial material.

With regard to Tab. 2, the “pulsatile compression” relates to rinsing the pericardial material three times for 60 s in isotonic NaCl solution while gently moving the pericardial material. Moreover, the pericardial material has been fully compressed with a stamp every five seconds for a short amount of time (e.g., for 1 s).

As shown in Tab. 2, the pulsatile compression of the biological material clearly leads to a reduction of remnant glutaraldehyde in the pericardial material. This may contribute to an avoidance of irreversible damages of the pericardial material in subsequent processing steps.

In step 611, the sponge-like pericardial material may be rinsed for 10-20 min, preferably for 15 min, in glycerin (with a concentration of 20-40%, preferably 30%) and ultrapure water as a stabilization step. During the rinsing step, the pericardial material may repeatedly be compressed with a compression time of 1-5 s, preferably 2 s and a decompression (relaxation time) of 1-10 s (preferably 5 s).

In step 612, the sponge-like pericardial material may be rinsed for 10-20 min, preferably for 15 min, in PEG-200 (with a concentration of 20-40%, preferably 40%) and ultrapure water as a stabilization step. During the rinsing step, the pericardial material may repeatedly be compressed with a compression time of 1-5 s, preferably 2 s and a decompression time (relaxation time) of 1-10 s (preferably 5 s).

In step 613, the sponge-like pericardial material may be rinsed for 10-20 min, preferably for 15 min, in PEG-400 (with a concentration of 20-40%, preferably 40%) and ultrapure water as a stabilization step. During the rinsing step, the pericardial material may repeatedly be compressed with a compression time of 1-5 s, preferably 2 s and a decompression time (relaxation time) of 1-10 s (preferably 5 s).

In step 614, the stabilized sponge-like material may further be dried in a climate cabinet. The biological material may preferably be placed on an impregnated material, impregnated with PEG-400 (with a concentration of 20-40%, preferably 40%) in an environment in which the relative humidity may be decreased from 90-99%, preferably from 95%, to 5-15%, preferably to 10% over 10-15 h, preferably over 12 h.

In step 615, the dried, sponge-like pericardial material may be placed between two sheets of a filter paper and may be compressed with a force of 74 kPa at room temperature for 1-5 min, preferably for 1 min. This last compression may supersede excess stabilizing solution from pores of the sponge-like material which may lead to a further reduction in thickness of the biological material. In some examples, for this final drying step (as outlined above), the pericardial material may preferably be placed on an impregnated material, wherein the impregnated material may preferably be impregnated with PEG-400 to avoid an unsatisfying stabilization of the pericardial material due to the extraction of stabilizing solution from the pericardial material (which may additionally be disadvantageously be intensified if the pericardial material was dabbed prior to the drying step).

The pericardial material obtained from the aforementioned process may be stored in the state of reduced thickness (and dried) until the pericardial material is used for a medical application.

It is further emphasized that the concentration of the stabilizing solutions, the compression force and the choice of the filter paper may be seen as the most crucial process parameters for the success of the manufacturing method of a biological material for a medical application. By the above-mentioned process a flexible biological material may be obtained which may be deformable (e.g., for being attached to an implant) in a dried state without cracking.

As a general remark, the obtained flexibility of the biological material may also depend on the initial thickness of the biological material. As an example, a dried sponge-like material with an initial thickness of approx. 2 mm may acquire a thickness of approx. 0.8 mm in a dried and compressed state. Even though such a material is still flexible and deformable, it may nevertheless be less flexible as compared to a biological material with a thickness of 0.1 mm. Also such smaller thickness materials may be provided.

Moreover, it is emphasized that any aforementioned compression step may also be replaced by a respective heat-pressing step. Heat pressing generally allows a variation of the viscosity of the stabilizing solution.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims

1. A method for preparing a porous biological material for a medical application, comprising:
exposing the biological material to a first liquid;
repeatedly applying a compression force onto the biological material over a plurality of compression intervals; and
drying the biological material.

2. The method according to claim 1, wherein the repeatedly applying comprises alternating compression intervals and at least one relaxation interval between ones of the compression intervals.

3. The method according to claim 2, wherein the at least one relaxation interval comprises 0.2 s to 30 s.

4. The method according to claim 3, wherein the compression intervals comprise 0.1 s to 10 s.

5. The method according to claim 1, wherein the repeatedly applying comprises a first compression step of applying a compression force in a range of 25 kPa to 500 kPa.

6. The method according to claim 5, comprising exposing the biological material to a second liquid after a first compression of the repeatedly compressing, wherein the second liquid comprises at least 10% of a component other than water and then applying a second compression of the repeatedly compressing.

7. The method according to claim 6, comprising exposing the biological material to a third liquid after the second compression, wherein the third liquid comprises at least 10% of a component other than water, and then applying a third compression of the repeatedly compressing.

8. The method according to claim 7, wherein the drying comprises drying the biological material on a material impregnated with the first, second and/or third liquid.

9. The method according to claim 1, wherein the repeatedly compressing is conducted to cause a reduction of a thickness of the biological material by 40% to 80%.

10. The method according to claim 1, wherein the first liquid comprises glycerin, and/or polyethylene glycol.

11. The method according to claim 1, wherein the biological material is a porous tissue sponge or a pericardial tissue sponge.

12. (canceled)

13. The method of claim 1, wherein the biological material is expandable to at least 70% of its initial thickness and surface area by re-hydration after the drying.

14. The method of claim 1, comprising applying the biological material after the drying to an implant.

15. The method of claim 14, comprising applying the biological material as a sealing element to the implant.
